(19) United States
(12) Patent Application Publication
(10) Pub. No.: US 2004/0097737 A1
(43) Pub. Date: May 20, 2004

(54) Use of phencynonate hydrochloride

(76) Inventors: Chuangui Liu, Beijing (CN); Qishan Liang, Beijing (CN); Zhanguo Gao, Beijing (CN); Wenyu Cui, Beijing (CN)

Correspondence Address: HARNESS, DICKEY & PIERCE, P.L.C., P.O. BOX 828, BLOOMFIELD HILLS, MI, 48303, US

(21) Appl. No.: 10/469,396

(22) PCT Filed: Feb. 28, 2002
(86) PCT No.: PCT/CN02/00132

(30) Foreign Application Priority Data
Feb. 28, 2001 (CN) 01104890.5

(51) Int. Cl. A61K 31/46; C07D 451/02

(52) U.S. Cl. 546/127; 514/304

(57) ABSTRACT

This invention relates to a new use of phencynonate hydrochloride, especially its use in the manufacture of medicament for treating or alleviating acute attack of vertigo.

### TECHNICAL FIELD

The present invention relates to new use of Phencynonate Hydrochloride, in particular, to its use in the manufacture of a medicament for treating or alleviating acute attack of vertigo.

### BACKGROUND ART

The structure of Phencynonate Hydrochloride, 2-phenyl-2-cyclopentyl -2-hydroxyacetic acid-3-methyl-3- azabicyclo(3, 3, 1) nonan-9 α-ester hydrochloride as the systematic name, is as follows:

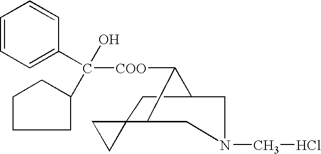

Chinese Patent applications Nos. 97125424.9 and 9311949491.1 disclose the preparation method and pharmaceutical compositions of Phencynonate Hydrochloride, as well as its use as an anti-motion sickness (such as car sickness, seasickness and airsickness, etc.) agent.

Vertigo is a disease induced by violent stimulation to the vestibular orgain. Usually, it is characterized by symptoms of lightheadedness, nausea and vomit, and difficulty in opening eyes and sitting still, etc. It is one of the common disorders in the E.N.T. department and Neurology department. It may be caused by various diseases, such as diseases of the ear (peripheral vertigo) or of the brain (central vertigo). Among them, Meniere's disease and positional vertigo have the highest incidence. Patients with Meniere's disease often go to see a doctor because of acute attack of vertigo. Currently, there is no effective drug available for this disease, and what doctors can do is to control the symptoms of acute attack with drug or only applying supportive therapy.

### OBJECT OF INVENTION

One object of this invention is to provide a drug that is useful in treating or alleviating acute attacks of vertigo.

### BRIEF DESCRIPTION OF THE INVENTION

The present inventors found that, Phencynonate Hydrochloride was more effective in treating or alleviating the acute attacks of vertigo than Diphenidol, a drug being commonly used in treating vertigo in clinical practice. From the observations on 231 patients who suffered from acute attacks of vertigo, the researchers confirmed this action of Phencynonate Hydrochloride.

In conclusion, this invention is directed to the use of Phencynonate Hydrochloride in treating or alleviating the acute attack of vertigo.

This invention also relates to a method of treating or alleviating patients with acute attack of vertigo, comprising administration of Phencynonate Hydrochloride to patients suffering from acute attack of vertigo.

This invention also relates to pharmacentical composition for treating or alleviating acute attack of vertigo, containing Phencynonate Hydrochloride and a pharnacentically vehicle or excipient.

### DETAILED DESCRIPTION OF THE INVENTION

The following Examples were intended to illustrate the invention in detail, without limiting the scope of the present invention in any way.

Patients, who came to out-patient department or emergency department and were diagnosed as acute attack of vertigo, were divided into two groups. One group received Phencynonate Hydrochloride, two tablets (2mg/tablet) as the first dose, followed by one tablet, twice or three times per day. The positive control group received Diphenidol, two tablets (25mg/tablet) as the first dose, followed by one tablet, twice or three times per day. Then the efficacy was observed for three days. It was considered as significantly effective when the symptom of vertigo disappeared, effective when the symptom is alleviated and no effect when there was no difference after taking the drug. The results are presented in table 1.

TABLE 1
Efficacy of Phencynonate Hydrochloride in treating acute vertigo

| | Phencynonate Hydrochloride Group | | | | Diphenidol Group | | | |
|---|---|---|---|---|---|---|---|---|
| | Sample | Significantly | | Total | Sample | Significantly | | Total |
| | size | effective | Effective | effective | size | effective | Effective | effective |
| Menicre's | 54 | 38 | 12 | 50* | 26 | 8 | 10 | 18 |
| disease | | (70.4) | (22.2) | (92.6) | | (30.8) | (38.4) | (69.2) |
| Positional | 13 | 12 | 1 | 13 | 3 | 1 | 1 | 2 |
| vertigo | | (92.3) | (7.7) | (100) | | (33.3) | (33.3) | (66.6) |
| Vertigo caused | 34 | 22 | 5 | 27 | 8 | 0 | 5 | 5 |
| by | | (64.7) | (14.7) | (79.4) | | (0) | (62.5) | (62.5) |
| vertebrobasilar | | | | | | | | |
| ischemia | | | | | | | | |
| Other vertigoes | 71 | 42 | 22 | 64 | 22 | 13 | 6 | 19 |
| | | (59.1) | (31.0) | (90.1) | | (59.0) | (27.3) | (86.3) |
| Subtotal | 172 | 114 | 40 | 154 | 59 | 22 | 22 | 44 |
| | | (66.3) | (23.3) | (89.6) | | (37.3) | (37.3) | (74.6) |

Total effective = Significantly effective + Effective
Phencynonate Hydrochloride vs. Diphenidol, *P < 0.05

As shown in table 1, there were 172 patients treated with Phencynonate Hydrochloride, and the total effective rate was 89.6%. Fifty-nine patients were treated with Diphenidol and the total effective rate was 74.6%. The total effective rate of Phencynonate Hydrochloride group was higher than that of control group, however, there was no significant difference between the two groups statistically (P>0.05). However, as for Meniere's disease, which is most commonly seen in clinical practice, the total effective rate of Phencynonate Hydrochloride was 92.6%, which was much higher than that of Diphenidol (69.2%). Statistical analysis revealed significant difference between the test and control groups (P<0.05). If only counting the samples of the Meniere's disease and Positional vertigo, the total effective percentage of Phencynonate Hydrochloride and Diphenidol was 94.0% (63/67) and 67.9%(19/28), respectively. Statistical analysis shows that the difference between test and control group was significant (P<0.01). In conclusion, Phencynonate Hydrochloride is effective at treating or alleviating the symptom of vertigo. In case of diseases with a higher incidence, the efficacy of Phencynonate Hydrochloride was significantly better than that of Diphenidol.

1. A use of phencynonate hydrochlorideof in the preparation of medicament for treating or alleviating acute symptom of vertigo. [chemical structure]
